(19) United States
(12) Patent Application Publication
(10) Pub. No.: US 2007/0191440 A1
(43) Pub. Date: Aug. 16, 2007

(54) Methods of treating autism and fragile X syndrome

(76) Inventors: Michael E. Solomon, Concord, MA (US); Randall L. Carpenter, Waban, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/654,968

(22) Filed: Jan. 18, 2007

Related U.S. Application Data
(60) Provisional application No. 60/762,153, filed on Jan. 25, 2006.

(51) Int. Cl. A61K 31/381

(52) U.S. Cl. 514/367

(57) ABSTRACT

Subjects having at least one condition selected from the group consisting of mental retardation, Down's syndrome, fragile X syndrome and autism are treated with a composition that includes Formula I.

### RELATED APPLICATION

This application claims the benefit of U.S. Provisional Application No. 60/762,153, filed on Jan. 25, 2006, the entire teachings of which are incorporated herein by reference.

### BACKGROUND OF THE INVENTION

Mental retardation, Down's syndrome, fragile X syndrome and autism are developmental and genetic disorders that affect day to day functioning, including learning, memory, speech, social skills and behavior. Currently available treatment regimens for humans with mental retardation, Down's syndrome, fragile X syndrome and autism to, for example, assist in day-to-day functioning, include behavioral modifications and treatment with a range of medications including anti-depressant and anti-psychotic drugs. However, such regimens frequently are not effective or may produce undesirable side-effects with long term use, particularly the use of anti-psychotic drugs. Thus, there is a need to develop new, improved and effective methods to treat mental retardation, Down's syndrome, fragile X syndrome and autism.

### SUMMARY OF THE INVENTION

The present invention relates to a method of treating a subject, comprising the step of administering to a subject having at least one condition selected from the group

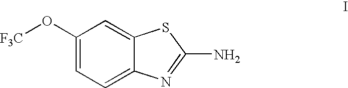

Advantages of the claimed invention can include, for example, treatment of mental retardation, Down's syndrome, fragile X syndrome and autism in a manner that can improve efficacy or reduce side effects and thereby improve tolerability for use over a relatively long period of time without significant side effects. The methods of the invention can provide an effective manner to treat a subject having mental retardation, Down's syndrome, fragile X syndrome and autism.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The invention includes a method of treating a subject, comprising the step of administering to a subject having at least one condition selected from the group consisting of mental retardation, Down's syndrome, fragile X syndrome and autism a composition that includes Formula I.

Formula I is 2-amino-6-trifluormethoxybenzothiazole and is also referred to herein as “riluzole” (see, for example, U.S. Pat. No. 4,370,338, the teachings of which are hereby incorporated by reference in its entirety).

Formula I (riluzole, 2-amino-6-trifluormethoxybenzothiazole) is FDA approved for the treatment of amyotrophic lateral sclerosis (ALS). It has multiple activities, including inhibition of presynaptic glutamate release by inactivation of P/Q-type calcium channels, enhancement of glutamate uptake in astrocytes, and inhibition of voltage-dependent sodium channels in mammalian CNS neurons. It has also been reported to potentiate AMPA/KA receptor-mediated transmission, as well as enhance brain-derived neurotrophic factor. Riluzole has been shown to have neuroprotective, anticonvulsant activity, anti-anxiety activity, and antidepressant activity in animal models and in humans (Mathew, J. S., et al., (2005)).

The subject can be treated with salts (e.g., HCl, oxaylate, salts of acids, calcium, sodium, magnesium, lithium), prodrugs and other structural and functional analogs, homologs, variants and derivatives thereof. Formula I.

Mental retardation means that a subject has lower than average intelligence. Intelligence describes a subject's ability to think, learn and solve problems. A subject with mental retardation may have difficulty learning, may take longer to learn social skills, such as how to communicate, and may be less able to care for himself or herself and to live on his or her own as an adult.

Down's syndrome is a disorder that includes a combination of birth defects, including some degree of mental retardation, characteristic facial features and, often, heart defects, increased infections, problems with vision and hearing, and other health problems. The severity of these problems varies greatly among affected subjects. Down's syndrome is generally is caused by an extra copy chromosome 21 and is also referred to as trisomy 21.

The fragile X syndrome, as implied by its name, is associated with a fragile site expressed as an isochromatid gap in the metaphase chromosome at map position Xq 27.3. Fragile X syndrome is a genetic disorder caused by a mutation in the 5′-untranslated region of the fragile X mental retardation 1 (FMR1) gene, located on the X chromosome. The mutation that causes fragile X syndrome is a associated with a CGG repeat in the fragile X mental retardation gene FMR-1. When a subject has more than about 200 CGG repeats, the fragile X gene is hypermethylated, silenced, fragile X mental retardation protein (FMRP) is not produced and the subject is diagnosed as having fragile X syndrome (See, for example, U.S. Pat. Nos. 6,107,025 and 6,180,337, the entire teachings of both of which are hereby incorporated by reference in their entirety).

Fragile X syndrome, the most common inherited form of mental retardation, is caused by a trinucleotide repeat expansion of the X-linked FMR1 gene that prevents expression of the encoded protein, fragile X mental retardation protein. Individuals with fragile X syndrome exhibit a number of symptoms, including mental retardation, developmental delay, attention deficit and hyperactivity, anxiety with mood liability, and obsessive-compulsive and autistic behaviors. People with fragile X syndrome also have increased incidence of epilepsy, poor coordination, heightened sensitivity to tactile irritation, and loose bowel movements. Non-neurological symptoms include a long face, large ears, hyperextensible joints, and enlarged testes in post-pubescent males. In addition, autopsies show that dendritic spines are longer and immature in appearance.

The fragile X syndrome segregates as an X-linked dominant disorder with reduced penetrance. Either sex when carrying the fragile X mutation may exhibit mental deficiency, which is variable in severity.

Children and adults with fragile X syndrome have varying degrees of mental retardation or learning disabilities and behavioral and emotional problems, including autistic-like features and tendencies. Young children with fragile X syndrome often have delays in developmental milestones, such as learning how to sit, walk and talk. Affected children may have frequent tantrums, difficulties in paying attention, frequent seizures (e.g., temporal lobe seizures) are often highly anxious, easily overwhelmed, can have sensory hyperarousal disorder, gastrointestinal disorders, may have speech problems and unusual behaviors, such as hand flapping and hand biting.

Fragile X syndrome can be diagnosed by an established genetic test performed on a blood sample from the subject. The test determines whether a mutation or pre-mutation is present in the FMR-1 gene of the subject.

Subjects with fragile X syndrome can also have autism, attention deficient disorder and/or obsessive compulsive disorder. Fragile X syndrome is a prevalent form of inherited mental retardation and is characterized by developmental delay, hyperactivity, attention deficit disorder and autistic-like behaviors (Jin, P., et al., Hum Mol Genet 9: 901-908 (2000)).

Subjects with autism can have several symptoms that can range from mild to severe. Such symptoms can included difficulties interacting with others; making friends; communication problems, both with spoken language and nonverbal gestures; insistence on sameness; and some degree of mental retardation or learning disabilities in most, but not all, of affected children. Subjects with a mild autistic spectrum disorder, referred to as Asperger syndrome, can share some of the features of autism, have normal intelligence and can learn to speak at the expected age. Autism is generally diagnosed by observing the behavior of the child and screening tests that assess a number of characteristics and behaviors associated with autism. Subjects with autism can also have, for example, obsessive compulsive behaviors, sleep disorders and/or gastrointestinal disorders.

The methods of the invention can be employed to treat additional conditions that can be associated with autism or fragile X syndrome, for example, Coffin-Lowry syndrome, Cohen syndrome, Duchenne/Becker muscular dystrophies, Neurofibromatosis, Joubert syndrome, Lujan-Fryns syndrome, PTEN mutations, Noonan syndrome, Orstavik syndrome, ARX mutations, CHARGE, Angelman syndrome, Nance-Horan syndrome, Prader-Willi syndrome, Cerebral dysgenesis and Smith-Lemli-Optiz syndrome.

The methods of the invention can be employed to treat pervasive developmental disorders with no obvious source, such as fragile X syndrome, mental retardation, Down's syndrome and autism and other disorders of brain development.

The methods of the invention can also be employed to treat disorders of brain development including Autism Spectrum Disorders (Pervasive Developmental Disorders), such as autism, fragile X syndrome, Down's syndrome, Rett's syndrome, Childhood Disintegrative Disorder, Asperger syndrome and Tuberous Sclerosis.

The methods of the invention can be employed to treat deficits/symptoms, for example, deficits in learning, memory, executive function, attention and/or processing speed. Such deficits can be deficits associated with or observed in subjects with mental retardation, fragile X syndrome, Down's syndrome and autism; and pervasive developmental disorders, including pervasive developmental disorders with no obvious source.

The methods of the invention can be employed to treat neuropsychiatric disorders and anxiety disorders, including anxiety disorders that are associated with or observed in subjects that have mental retardation, autism, Down's syndrome and fragile X syndrome. Such anxiety disorders include, for example, specific phobias, such as phobias of the doctor and dentist; agoraphobia and separation anxiety. Such disorders can also include, for example, depression, bipolar disorders, repetitive and stereotyped behavior, obsessive and compulsive traits/disorders, aggressive behavior, schizophrenia, hyperactivity, pain, itching, sensory hyperarousal, seizures, behavioral problems, sleep disorders (including insomnia, hypersomnia and abnormal behaviors during sleep).

The methods of the invention can also be employed to treat gastrointestinal disorders and metabolic disorders in subjects with mental retardation, fragile X syndrome, Down's syndrome and autism. Autistic behavior (deficits in social interaction, verbal and non-verbal communication, and restricted/repetitive behaviors or interests) in subjects with autism, mental retardation, fragile X syndrome and Down's syndrome can also be treated by the methods of the invention.

One skilled in the art would be able to assess the disorders or conditions described above (See, for example, Patzold, L. M., et al., J. Paediatr. Child Health, 34:528-533 (1998); Malow, B. A., Ment. Retard Dev. Disabil. Res. Rev. 10:122-125 (2004); Robinson, A. M., et al., Child Care Health Dev. 30:139-150 (2004); Couturier, J. L., et al., J. Am. Acad Child Adolesc Psychiatry 44:815-822 (2005); Kuddo, T., et al., Curr. Opin. Pediatr. 15:339-343 (2003); Molloy, C. A., et al., Autism 7:165-171 (2003)).

The methods of the invention can be employed to treat fragile X-associated tremor/ataxia syndrome (FXTAS) and movement disorders. As discussed above, an excess of 200 CGG repeats in the 5′-untranslated region of the FMR1 gene results in transcriptional silencing of the FMR1 gene and fragile X syndrome. Subjects with permutation expansions (about 55 to about 200 CGG repeats in the FMR1 gene) are generally unaffected intellectually and may develop FXTAS, which is characterized by progressive cerebellar ataxia, parkinsonism, dementia and autonomic dysfunction (Baba, Y., et al., Current Opinion in Neurology 18:393-398 (2005), the teachings of which are hereby incorporated by reference in its entirety).

Activation of Group I metabotropic receptors induces protein synthesis, which is dependent on mRNA translation. This protein synthesis results in long-term depression (LTD), a form of synaptic plasticity. In mice lacking FMRP, mGluR induced protein synthesis and corresponding long-term depression is increased in the hippocampus. FMRP may act as a repressor of protein synthesis and is required for stable expression of mGluR-dependent LTD. Many lasting consequences of Group I mGluR activation may require protein synthesis and which are exaggerated in the absence of FMRP. Thus fragile X syndrome may be treated by inhibiting the activation of the Group I receptors and/or the downstream intracellular signals they initiate.

Riluzole, can inhibit glutamate release and promote glutamate uptake, and may be effective for treatment of the symptoms of fragile X syndrome, autism, and mental retardation. By inhibiting the release of glutamate and/or promoting the uptake of glutamate, the pool of glutamate that can activate the Group I metabotropic receptors is decreased. Thus, it can indirectly inhibit activation of Group I metabotropic receptors and thereby treat fragile X syndrome, autism and mental retardation.

One of skill in the art would be able to employ well-established criteria to diagnosis a subject that has mental retardation, Down's syndrome, fragile X syndrome and autism and the conditions or deficits described herein.

The subject treatment by the methods of the invention described herein can be a rodent (e.g., mouse, rat) or a primate (e.g., a monkey, baboon, human). In a particular embodiment, the subject is a human.

The compounds employed in the methods of the invention can be administered to the subject acutely (briefly or short-term) or chronically (prolonged or long-term).

An “effective amount,” also referred to herein as a “therapeutically effective amount,” when referring to the amount of a compound or composition (2-amino-6-trifluormethoxybenzothiazole) is defined as that amount, or dose, of a compound or composition that, when administered to a subject, is sufficient for therapeutic efficacy (e.g., an amount sufficient decrease to exhibit a clinical improvement in a behavior or mental cognitive test score; alleviate sensory hyperarousal disorder, an anxiety disorder, a seizure disorder, a gastrointestinal disorder, a sleep disorder, prevent weight gain, decrease obsessive compulsive tendencies and manners).

The methods of the present invention can be accomplished by the administration of the compounds of the invention (e.g., compositions including 2-amino-6-trifluormethoxybenzothiazole) by enteral or parenteral means. The route of administration can be by oral ingestion (e.g., tablet, capsule form) or intramuscular injection of the compound. Other routes of administration can include intravenous, intraarterial, intraperitoneal, or subcutaneous routes, nasal administration, suppositories and transdermal patches.

The compounds (e.g., 2-amino-6-trifluormethoxybenzothiazole) employed in the methods of the invention can be administered in a dose of between about 0.1 mg/kg to about 1 mg/kg body weight; about 1 mg/kg to about 5 mg/kg body weight; or between about 5 mg/kg to about 15 mg/kg body weight. The compounds can be administered in doses of about 0.1 mg, about 1 mg, about 2 mg, about 10 mg, about 25 mg, about 50 mg, 100 mg, about 200 mg, about 250 mg, about 300 mg, about 350 mg, about 400 mg, about 500 mg, about 600 mg, about 700 mg, about 900 mg, about 1000 mg, about 1200 mg, about 1400 mg, about 1600 mg or about 2000 mg. The compounds can be administered once a day or multiple (e.g., two, three, four, five) times per day.

The compounds employed in the methods of the invention can be administered alone or can be coadministered to the patient. Coadminstration is meant to include simultaneous or sequential administration of one or more of the compounds employed in the methods of the invention individually or in combination. The mode of administration can be conducted sufficiently close in time to each other so that the effects on the subject are maximal. It is also envisioned that multiple routes of administration (e.g., intramuscular, oral, intranasal, inhalation, topical, transdermal) can be used to administer the compounds employed in the methods of the invention.

The compounds employed in the methods of the invention can be administered alone or as admixtures with conventional excipients, for example, pharmaceutically, or physiologically, acceptable organic, or inorganic carrier substances suitable for enteral or parenteral application which do not deleteriously react with the compound(s) administered to the subject. Suitable pharmaceutically acceptable carriers include water, salt solutions (such as Ringer's solution), alcohols, oils, gelatins and carbohydrates such as lactose, amylose or starch, fatty acid esters, hydroxymethylcellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compounds employed in the methods of the invention. The preparations can also be combined, when desired, with other active substances to reduce metabolic degradation. A preferred method of administration of the compounds employed in the methods of the invention can be oral administration, such as a tablet.

The compounds employed in the methods of the invention, alone, or when combined with an admixture, can be administered in a single or in more than one dose over a period of time to confer the desired effect (e.g., alleviate symptoms of autism, improve sleep patterns, decrease sensory hyperarousal disorder, alleviate an anxiety disorder, a seizure disorder, a gastrointestinal disorder, an impaired cognitive function, weight gain).

When parenteral application is needed or desired, particularly suitable admixtures for the compounds employed in the methods of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds for use in the methods of the invention can also be incorporated into liposomes or administered by transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of which are hereby incorporated by reference.

The dosage and frequency (single or multiple doses) administered to an individual can vary depending upon a variety of factors, including the duration of condition of the subject (e.g., sensory hyperarousal disorder, anxiety disorder, seizure disorder, gastrointestinal disorder, sleep disorder, an impaired cognitive function, weight gain, obsessive compulsive behaviors); the route of administration of the compound; size, age, sex, health, body weight, body mass index, and diet of the recipient; nature and extent of symptoms of the disorder being treated (e.g., sensory hyperarousal disorder, anxiety disorder, seizure disorder, gastrointestinal disorder, sleep disorder, impaired cognitive function), kind of concurrent treatment (e.g., behavioral modification, anti-depressant medications, ÿ-adrenergic agonists, anticonvulsants, a nicotinic receptor agonist, an endocannabinoid receptor antagonist, AMPA agonists, anti-psychotics), complications from, for example, a sensory hyperarousal disorder, anxiety disorder, seizure disorder, gastrointestinal disorder, sleep disorder or impaired cognitive function; or other health-related problems. Other therapeutic regimens or agents can be used in conjunction with the methods of the present invention. For example, the administration of the compounds employed in the methods of the invention can be accompanied by behavioral modifications, anti-depressant medications and anti-psychotic medications. Adjustment and manipulation of established dosages (e.g., frequency and duration) are well within the ability of those skilled in the art.

In one embodiment, the antipsychotic compound employed in the methods of the invention can be a typical antipsychotic compound (also referred to as “a typical antipsychotic agent” or a “typical antipsychotic drug”). In another embodiment, the antipsychotic compound is an atypical antipsychotic compound (also referred to as an “atypical antipsychotic agent,” an “atypical antipsychotic drug” or a “second generation antipsychotic”).

Exemplary atypical antipsychotic compounds for use in the methods of the invention can be at least one member selected from the group consisting of zuclopenthixol, amisulpride, aripiprazole (7-[4-[4-(2,3-dichlorophenyl)-1-piperazinyl]butoxy]-3-4-dihydrocarbostyril), nemonapride, abaperidone (7-[3-[4-(6-fluoro-1,2-benzisoxazol-3-yl)-1-piperidinyl]propoxy]-3-(-hydroxymethyl)-4H-1-benzopyran-4-one, U.S. Pat. No. 5,736,588, the teachings of which are hereby incorporated by reference in its entirety; belaperidone ((1.alpha.,5.alpha.,6.alpha.)-3-[2-[6-(4-fluorophenyl)-3-azabicyclo[-3.2.0]-hept-3-yl]ethyl]-2,4(1H,3H)quinazolinedione, U.S. Pat. No. 5,475,105, the teachings of which are hereby incorporated by reference in its entirety; clozapine (8-chloro-11-(4-methyl-1-piperazinyl)-5H-dibenzo[b,e][1,4]diazepine, U.S. Pat. No. 3,539,573, the teachings of which are hereby incorporated by reference in its entirety issued; iloperidone (1-[4-[3-[4-(6-fluoro-1,2-benzisoxazol-3-yl)-1-piperidinyl]-3-methoxy-phenyl]ethanone; EP-402,644, the teachings of which are hereby incorporated by reference in its entirety; olanzapine (2-methyl-4-(4-methyl-1-piperazinyl)-10H-thieno[2,3-b][1,5]benzodiazepine; U.S. Pat. No. 5,229,382, the teachings of which are hereby incorporated by reference in its entirety; perospirone(cis-2-[4-[4-(1,2-benzisothiazol-3-yl)-1-piperazinyl]butyl]-hexahydro-o-1H-isoindole-1,3(2H)-dione, U.S. Pat. No. 4,745,117, the teachings of which are hereby incorporated by reference in its entirety; risperidone (3-[2-[4-(6-fluoro-1,2-benzisoxazol-3-yl)piperidino]ethyl]-2-methyl-6,7,8,9-tetrahydro-4H-pyrido[1,2-.alpha.]pyrimidin-4-one), U.S. Pat. No. 4,804,663, the teachings of which are hereby incorporated by reference in its entirety; sertindole (1-[2-[4-[5-chloro-1-(4-fluorophenyl-1H-indol-3-yl]-1-piperidinyl]eth-yl]imidazolidin-2-one), U.S. Pat. Nos. 4,710,500; 5,112,838; and 5,238,945, the teachings of all of which are hereby incorporated by reference in their entirety; tiospirone (8-[4-[4-(1,2-benzisothiazol-3-yl)-1-piperazinyl]butyl]-8-azaspiro[4-0.5]decane-7,9-dione), U.S. Pat. No. 4,411,901, the teachings of which are hereby incorporated by reference in its entirety; ziprasidone (5-[2-[4-(1,2-benzoisothiazole-3-yl)-1-piperazinyl]ethyl]-6-chloro-1-,3-dihydro-2-one), U.S. Pat. No. 4,831,031, the teachings of which are hereby incorporated by reference in its entirety; zotepine (2-[(8-chlorodibenzo[b,f]thiepin-10-yl)oxy]-N,N-dimethyl-ethanamine), U.S. Pat. No. 3,704,245, the teachings of which are hereby incorporated by reference in its entirety; quetiapine (5-[2-(4-dibenzo[b,f][1,4]thiazepin-11-yl-1piperazinyl)ethoxy]ethano-1), U.S. Pat. No. 4,879,288, the teachings of which are hereby incorporated by reference in its entirety; and blonanserin (2-(4-ethyl-1-piperazinyl)-4-(4-fluorophenyl)-5,6,7,8,9,10-hexahydro-1-cycloocta[b]pyridine), U.S. Pat. No. 5,021,421, the teachings of which are hereby incorporated by reference in its entirety; 2002/0123490, the teachings of which are hereby incorporated by reference in its entirety).

Antipsychotic agents, including atypical antipsychotic compounds for use in the invention can include, for example, Acetophenazine Maleate; Alentemol Hydrobromide; Alpertine; Azaperone; Batelapine Maleate; Benperidol; Benzindopyrine Hydrochloride; Brofoxine; Bromperidol; Butaclamol Hydrochloride; Butaperazine; Carphenazine Maleate; Carvotroline Hydrochloride; Chlorpromazine; Chlorprothixene; Cinperene; Cintriamide; Clomacran Phosphate; Clopenthixol; Clopimozide; Clopipazan Mesylate; Cloroperone Hydrochloride; Clothiapine; Clothixamide Maleate; Clozapine; Cyclophenazine Hydrochloride; Droperidol; Etazolate Hydrochloride; Fenimide; Flucindole; Flumezapine; Fluphenazine Decanoate; Fluphenazine Enanthate; Fluphenazine Hydrochloride; Fluspiperone; Fluspirilene; Flutroline; Gevotroline Hydrochloride; Halopemide; Haloperidol; Iloperidone; Imidoline Hydrochloride; Lenperone; Loxapine; Mazapertine Succinate; Mesoridazine; Metiapine; Milenperone; Milipertine; Molindone Hydrochloride; Naranol Hydrochloride; Neflumozide Hydrochloride; Ocaperidone; Olanzapine; Oxiperomide; Penfluridol; Pentiapine Maleate; Perphenazine; Pimozide; Pinoxepin Hydrochloride; Pipamperone; Piperacetazine; Pipotiazine Palmnitate; Piquindone Hydrochloride; Prochlorperazine Edisylate; Prochlorperazine Maleate; Promazine Hydrochloride; Quetiapine; Remoxipride; Quetiapine Remoxipride Hydrochloride; Risperidone; Risperadone Rimcazole Hydrochloride; Seperidol Hydrochloride; Sertindole; Setoperone; Spiperone; Sulpiride; Thioridazine; Thiothixene; Thorazine; Tioperidone Hydrochloride; Tiospirone Hydrochloride; Trifluoperazine Hydrochloride; Trifluperidol; Triflupromazine; Ziprasidone Hydrochloride, analogs, derivative and combinations thereof (see, for example, U.S. Patent Application Nos: 20040019030 and 2 002/0123490, the teachings of both of which are hereby incorporated by reference in their entirety).

Antipsychotic compounds can have adverse side effects including, for example, central nervous system depression, weight gain, sexual dysfunction, adverse effects on mood, anticholinergic side effects (cognitive impairment, reduced memory capacity, confusion, delirium, dry mouth, blurred vision, worsening of glaucoma, constipation, urinary retention, tachycardia), weight gain, diabetes mellitus, prolactin elevation, QTC prolongation, sedation, motor side effects such as extrapyramidal symptoms (EPS), dystonia, drug-induced parkinsonism, akathisia and potentially persistent drug-induced movement disorders and motor side effects such as tardive dyskinesia (see, for example, U.S. Publication No: 2003/0008897, the teachings of which are hereby incorporated by reference in its entirety). These adverse side effects can reduce patient compliance and lead to relapses.

Atypical antipsychotic compounds can reduce psychotic symptoms with fewer side effects (e.g., extrapyramidal side effects, rigidity, tremor, akathisia, cognitive impairment) than typical antipsychotics (see, for example, Citrome, L., et al., Postgraduate Medicine 116: (2004)). In addition, atypical antipsychotics can also reduce aggression, repetitive behaviors, hallucinations, delusions, amotivation and emotional withdrawal. However, not all side effects (e.g., weight gain, impaired glucose tolerance/lipid abnormalities, impaired social interaction) are eliminated by the use of atypical antipsychotics. Group I mGluR antagonist have been shown to reduce weight gain and decrease appetite. Combinations of Group I mGluR antagonists and antipsychotics in the methods of the invention described herein, in particular atypical antipsychotics, may diminish or reduce the side effects of antipsychotic compounds y reducing the dosage required and increase compliance to thereby treat subjects having conditions such as mental retardation, fragile X syndrome, Down's syndrome, autism, pervasive developmental disorders, including pervasive developmental disorders with no obvious source.

An “agonist,” as used herein, is a compound that activates cell signaling. For example, an ÿ-adrenergic agonist activates cell signaling mediated through ÿ-adrenergic receptors.

The compounds employed in the methods of the invention can be administered to a subject with (e.g., before, concomitantly, sequentially or after) administration of other compounds that are employed to treat a particular disorder or condition in the subject. For example, the compositions of the invention can be administered with at least one member selected from the group consisting of an antidepressant, an anti-psychotic, an ÿ-adrenergic agonist, an anticonvulsant, a nicotinic receptor agonist, an endocannabinoid receptor antagonist and an AMPA agonist.

The identification of appropriate compounds, such as antidepressants, antipsychotics, ÿ-adrenergic agonists, anticonvulsants, a nicotinic receptor agonist, an endocannabinoid receptor antagonist and AMPA agonists, for use in the methods of the invention would be known to one skilled in the art (see, for example, Beryy-Kravis, E., et al., Mental Retardation and Developmental Disabilities 10: 42-48 (2004), the teachings of which are hereby incorporated by reference in its entirety).

### EQUIVALENTS

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a subject, comprising the step of administering to a subject having at least one condition selected from the group consisting of mental retardation, Down's syndrome, fragile X syndrome and autism a composition that includes Formula I.

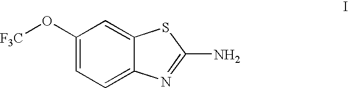

2. The method of claim 1, wherein the subject is a human.

3. The method of claim 1, wherein the human has fragile X syndrome.

4. The method of claim 1, wherein the subject has autism.

5. The method of claim 3, wherein the human further has autism.

6. The method of claim 1, further including the step of administering at least one member selected from the group consisting of an antidepressant, an ÿ-adrenergic agonist, an anticonvulsant, a nicotinic receptor agonist, an endocannabinoid receptor agonist, and anticonvulsant, an atypical anti-psychotic and an AMPA agonist.

7. The method of claim 1, wherein the human further has at least one condition selected from the group consisting of a sensory hyperarousal disorder, an anxiety disorder, a seizure disorder, a gastrointestinal disorder, a sleep disorder and an impaired cognitive function.
